(19) United States
(12) Patent Application Publication
(10) Pub. No.: US 2016/0145557 A1
(43) Pub. Date: May 26, 2016

(54) Reactor vessel useful for performing multiple pretreatments

(71) Applicant: The Regents of the University of California, Oakland, CA (US)

(72) Inventors: Deepti Tanjore, Berkeley, CA (US); Joseph Rasson, Danville, CA (US); James Gardner, Oakland, CA (US); Paul Perry, Los Gatos, CA (US); Akash Narani, Emeryville, CA (US); Chenlin Li, Castro Valley, CA (US)

(73) Assignee: THE REGENTS OF THE UNIVERSITY OF CALIFORNIA, Oakland, CA (US)

(21) Appl. No.: 14/944,036

(22) Filed: Nov. 17, 2015

Related U.S. Application Data
(60) Provisional application No. 62/080,968, filed on Nov. 17, 2014.

(51) Int. Cl. C12M 1/42; B01J 8/00; B01J 8/02

(52) U.S. Cl. CPC C12M 35/04; B01J 8/02; B01J 8/0005; B01J 2208/00823

(57) ABSTRACT

The present invention provides for a reactor vessel comprising a reaction chamber comprising an inner surface configured for performing multiple pretreatments.

### RELATED PATENT APPLICATIONS

The application claims priority to U.S. Provisional Patent Application Ser. No. 62/080,968, filed Nov. 17, 2014; which is incorporated herein by reference.

### STATEMENT OF GOVERNMENTAL SUPPORT

The invention was made with government support under Contract No. DE-AC02-05CH11231 awarded by the U.S. Department of Energy. The government has certain rights in the invention.

### FIELD OF THE INVENTION

The present invention is in the field of pressure reactors.

### BACKGROUND OF THE INVENTION

A sustainable source for generation of renewable liquid fuels is lignocellulosic biomass, mostly agricultural and forest residues. Conversion of lignocellulosic biomass to liquid fuels is challenging because biomass and its two main polymers, lignin and glucan, are insoluble in water and several other types of solvents, especially at room temperature. Catalysis of insoluble biomass is necessary to separate and depolymerize the polymers, primarily, lignin, glucan, and xylan.

Researchers have developed several biomass depolymerization technologies, including thermochemical (homogenous and heterogeneous) and biological (enzymatic) treatments, to break the insoluble cross-linked matrix of lignin and recover sugar-rich polymers: xylan and insoluble glucan. These polymers are further chemically and/or enzymatically broken down to soluble monomeric sugars or other intermediates in a separate unit operation, mostly after being transferred into a different vessel. Biorefineries can economically ferment these monomeric sugars or further catalyze the intermediates to several biochemicals and fuels through traditional and emerging technologies. Since biomass is the single largest cost in bio-ethanol production, to establish an economical pathway for biofuel production, biorefineries should focus on maximizing biomass depolymerization to sugars and other intermediates. To achieve this goal, several research labs across the country have developed technologies at small scale with a single or combination of a broad range of catalysts (including concentrated and dilute acids, steam, ammonia, organic solvents, alkalis, ionic liquids, and solid acid catalysts, etc.) at high temperatures and pressures, typically above 150° C. and 200 psi in single and multiple phase treatments. Sometimes, researchers follow up these thermochemical processes with enzymatic treatments (conducted at 50° C. and atmospheric pressure) to produce monomeric sugars for downstream fermentation. Previous studies suggest that no single thermochemical treatment may efficiently process all reactant types (hardwoods, softwoods, herbaceous, agricultural residues, municipal solid wastes, residues from pulp and paper industries, etc.) To avoid loss in process yields and performance due to the generation of inhibitory compounds, researchers designed unique thermochemical reactors best suited to specific combinations of chemical conditions and feedstocks.

The available and established reactor designs for several thermochemical technologies are disparate with no single system to accommodate several, if not all, technologies and feedstocks. Moreover, most of these reactor designs do not include mixing, which is an essential process condition for enzymatic hydrolysis, especially at high solids loading. High solids loading are necessary for economical production of fuels or chemicals in commercial facilities. Even if a chemical pathway to convert soluble glucose at low solids loading was to be pursued, latest studies suggest the application of solid acid catalyst that can be recovered and recycled. However, the aqueous phase should be mixed continuously to keep the catalyst suspended in the aqueous phase. A reactor that can perform the requirements listed above has not yet been designed.

Current reactors do not have any of the following features: that suggest any attempts at designing a reactor for integration of multiple catalytic technologies (homogenous and heterogenous; chemical and biological) in a single vessel for all insoluble feedstocks at high solids loadings. Most facilities, including small scale universities and large scale national laboratories and companies, are performing a single thermochemical treatment unit operation that may or may not be followed by another chemical or enzymatic treatment in a separate vessel. No facility provides several thermochemical (homogenous and heterogenous) as well as enzymatic treatment in a single reactor at large scale (100 L working volume) for insoluble reactants. Also, there is no reactor that can perform both upstream deconstruction and downstream chemical catalysis.

### SUMMARY OF THE INVENTION

The present invention provides for a reactor vessel comprising a reaction chamber comprising an inner surface configured for performing multiple pretreatments. In some embodiments, the reactor vessel is configured for integration of multiple catalytic technologies in a single vessel for an insoluble feedstock at high solids loadings. The reactor vessel can comprise one or more features described herein. The reactor vessel is useful for performing multiple thermochemical technologies and enzymatic hydrolysis in a single vessel. In some embodiments, the reactor vessel is a pressure reactor. In some embodiments, the reactor vessel is a single-stirred pressure reactor.

The reactor vessel is configured for integration of multiple catalytic technologies (homogenous and heterogeneous; chemical and biological) in a single vessel for all insoluble feedstocks at high solids loadings. In some embodiments, the reactor vessel is configured to perform one or more thermochemical (homogenous and/or heterogeneous) and enzymatic treatments in a single reactor at large scale (at least 100 L working volume) for insoluble reactants.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows a simple schematic of a batch high-pressure/temperature vessel to perform homogenous and heterogeneous catalysis of soluble or insoluble reactants in an aqueous phase with or without vacuum or a gaseous overlay and with or without direct or indirect heating.

FIG. 2 shows the heating profile of water using an embodiment of the invention.

FIG. 3 shows the heating profile for insoluble biomass in acidic water (pH<3.0) using an embodiment of the invention.

FIG. 4 shows an exemplary commissioning process and results.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used in the specification and the appended claims, the singular forms “a”, “an”, and “the” include plural references unless the context clearly dictates otherwise. Thus, for example, reference to a “reactant” includes a single reactant as well as a plurality of reactants.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” refers to a value including 10% more than the stated value and 10% less than the stated value.

These and other objects, advantages, and features of the invention will become apparent to those persons skilled in the art upon reading the details of the invention as more fully described below.

In some embodiments, the reactor vessel comprises one or more of the following features described herein.

In some embodiments, the reactor chamber has a volume of from about 0.2 L to about 300 L. In some embodiments, the reactor chamber has a volume of about 0.2 L, about 2 L, about 10 l, about 50 L, or about 300 L.

In some embodiments, the reactor chamber is configured from an ILA reactor. In some embodiments, the reactor chamber is configured from a Parr reactor, such as a TCuP reactor, such as a Parr Reactor 4557. In some embodiments, the reactor chamber is configured from an Andritz reactor, such as a TCuP reactor, such as an Andritz Helical Dryer. In some embodiments, the reactor chamber is configured from an ABEC reactor. In some embodiments, the reactor chamber is configured from a BioEngineering Fermenter reactor.

In some embodiments, the reactor chamber comprise one or more of the features described in Table 1.

TABLE 1

| Features/ | | |
|---|---|---|
| Capabilities | Parr Pressure Reactor | Andritz Helical Dryer |
| Material of | Hastelloy C-276 | Hastelloy C-22 |
| Construction | (Customized) | (Customized) |
| Maximum Working | 6 liters | 100 liters |
| volume | | |
| Operating | 0-350° C., 1800 psig | 175° C., 150 psig |
| conditions | | (Customized) |
| Impeller | Anchor w/PTFE blades | Helical shaped |
| Direct heating | Fitted w/steam or heated | Fitted w/steam or heated |
| | gaseous catalyst injection | gaseous catalyst injection |
| | port (Customized) | port (Customized) |
| Indirect heating | Electric band heater | Heated oil through jacket |

In some embodiments, the reactor vessel can handle high concentrations of insoluble solids in the substrate. In some embodiments, the reactor vessel, such as the Parr Reactor, has one or more of the following features: pressure gauge, steam injection line/valve, pressure sensor, condensate relief valve/line, thermocouple, burst disk pressure relief, gas inlet valve/port, pressure relief valve, three-way valve, burst disk pressure relief, and liquid injection port. In some embodiments, the reactor vessel, such as the Andritz Pressure Vessel Reactor, has one or more of the following features: gear box, motor, feed port, catalyst inlet line, to—TCU oil return, from—TCU oil supply, steam supply line, RODI valve, sight glass, air cylinder, and discharge valve. In some embodiments, the reactor vessel, such as the Andritz Pressure Vessel Reactor, has one or more of the following features: discharge valve opens under pressure, head space for solvent expansion, and mass flow meters to measure solvent input and loss. In some embodiments, the reactor vessel, such as the Parr Reactor, has one or more of the following features: magnetic drive and self-sealing packing gland drive.

In some embodiments, the reactor vessel comprises an impeller and one or more means of direct and/or indirect heating of the reaction chamber. In some embodiments, the impeller is an anchor impeller, helical impeller, or turbine impeller. In some embodiments, the impeller is configured to close to the inner surface of the reaction chamber. In some embodiments, the impeller is capable of impelling a mixture or suspension having a viscosity of from 10⁴ to 10⁸ cP. The mixture or suspension comprises one or more insoluble reactants. In some embodiments, the insoluble reactant is lignocellulosic biomass, hardwood, softwoods, herbaceous, agricultural residues, municipal solid waste, residues from pulp and paper industries, and the like, and mixtures thereof. In some embodiments, direct heating is rapid heat-up to target reaction temperature, which is especially important for high solids loadings. This provides uniform temperature distribution throughout the reactor. In some embodiments, indirect heating media for scale up reactors are used. Suitable media are steam, hot air, hot compressed air and hot compressed carbon dioxide. Like steam injection (as direct heating) can increase the temperature of the reactor contents to the reaction temperature at a faster rate. Disadvantages of adding direct steam can comprise: challenge to achieve high solids concentration after pretreatment/reaction due to dilution with steam condensate, and achieving accurate material balance closures is more challenging because of loss of volatile components in the flash stream. In some embodiments, the impeller can handle viscosities from 10⁴ to 10⁸ cP.

In some embodiments, the anchor impeller comprises: three arm, self-centering anchor with PTFE wiper blades, this works well for blending and maximum heating transfer from the reactor jacket to the slurry. They are suitable for moderate to high viscosities (10⁴ to 10⁵ cP), and for uniform mixing and there is no temperature gradient. Operation at slow speeds can require a heavy duty drive system capable of generating sufficient torque to the agitator (Gear drive system). In some embodiments, the turbine impeller works really well for avicel and IL loading, and is excellent mixing for systems with effective viscosities of 25,000 cP. In some embodiments, the helical impeller comprises: a blade that travels close to the wall of the tank to force good overall circulation, and is efficient mixing of dry materials. Sticky products can be processed thanks to a special design of the mixing screw.

The design or configuration of a reactor vessel for thermochemical treatments of insoluble reactants is unique to each catalyst because the optimum or appropriate operating conditions (reaction temperature, pressure, and mixing) can vary significantly. For example, for the depolymerization of lignocellulosic biomass, literature suggests hydrogen peroxide pretreatments can be performed optimally at atmospheric pressure, below 100° C. with mixing, whereas ammonia fiber expansion (AFEX) should be conducted without any mixing at much higher temperatures and pressures, i.e., 120° C. at 600 psi. In some cases, such as steam explosion (250 psi at 180° C.), the catalyst itself acts as a mode of heat transfer. Researchers have been successful in performing hydrothermal (no catalyst except water) pretreatments with high solids by injecting steam into biomass at high pressure (150-250 psi) in a “steam gun” to maintain water in liquid form at high temperatures (160-200° C.) for a very short reaction time of 5 to 10 minutes. Longer heating or reaction times can lead to inhibitor production during dilute acid or hydrothermal pretreatments. Compared to other established technologies, such as AFEX, the heat up and cool down ramps of dilute acid and hydrothermal pretreatments should be much faster and accordingly the reactor design involves heating of biomass through the catalyst itself, rather than from an external source, such as a heating jacket. However, this steam gun reactor design, similar to AFEX reactor design, does not include mixing. Due to the short reaction times efficient mass transfer of catalyst and heat transfer to the entire biomass load is essential. Mixing biomass can improve mass and heat transfer, which can otherwise prove to be an obstacle for economical execution of the technology at large scale with high solids loading (−30% w/w biomass). Apart from homogenization, shear caused from mixing can act as a mild mechanical pretreatment itself. Such mild pretreatment will allow the operator to perform the thermochemical pretreatment at a lower temperature leading to lower inhibitor production and environmental and safety hazards. Accordingly, a single pressure reactor with mixing and fast heat up and cool down ramps can accommodate the execution of several pretreatment technologies and enzymatic hydrolysis on all feedstocks that will favorably result the following: (i) improve upstream pretreatment process yields due to better mass and heat transfer, (ii) reduce environmental and safety hazards due to lower reaction temperatures, (ii) reduce inhibitor production and biomass loss to inhibitor production due to better mass and heat transfer, (iv) improve downstream fermentation process yields due to lower inhibitor yields, (iii) improve overall process yields due to reduction in the number of vessels. The reactor vessel can comprise one or more of the features described above. In some embodiments, the biomass is about 15% (w/w) lignocellulosic biomass.

The reactor vessel can be modified from certain off-the-shelf vessels are commercially available. The reactor vessel can be configured to comprise one or more of the following: different mixing mechanisms, rapid heat up or cool down ramps, and pressure handling capability. In some embodiments, the reactor vessel is configured to perform multiple pretreatments followed by enzymatic hydrolysis in a single vessel for all feedstocks to improve flexibility in the acceptance of feedstock by a biorefinery to process for biofuel production.

In some embodiments, the reactor vessel is configured to enable a researcher to evaluate the scalability issues associated with novel and/or established small scale thermochemical and/or enzymatic treatments. Through this reactor design, production scale facilities can be flexible in accepting and processing several feedstock types by performing multiple established thermochemical and enzymatic treatments. Faster temperature and pressure ramp rates using multiple chemical treatments will improve yields (e.g. sugar from lignocellulosic biomass) and reduce inhibitor (e.g. furfural and hydroxymethylfurfural (HMF) from lignocellulosic biomass) yields. Also, for the case of lignocellulosic biomass, studies have indicated that lignin can also act as an inhibitor during fermentation. Performing hydrolysis of feedstocks to simpler sugars and separating lignin from biomass prior to fermentation will improve overall process yields and provide a possibility to vend lignin as a co-product.

In some embodiments, the reactor vessel is configured to be highly valuable to biorefineries for evaluating several thermochemical and enzymatic treatments of multiple feedstocks at high solid loadings in a single reactor to produce and separate liquor with high concentration of product(s) and low concentration of inhibitory compounds. Facilitated by the improved mass and heat transfer in this design, the kinetics, concentrations, and yields of feedstock conversion will be more easily controlled and optimized at a higher level. We developed a system that will:

In some embodiments, the reactor vessel is configured to comprises one or more of the following features: (1) inject catalyst (e.g. steam, ammonia, liquid catalysts, and/or solvents) into the pressure reactor through separate ports with mass flow meters while mixing the biomass in the reactor to establish efficient addition of liquid and gaseous catalysts; (2) allow injection of preheated catalyst through a port with mass flow meter, for direct and rapid heat up; (3) release insoluble reactants rapidly through a valve that can open under pressure to achieve explosion level treatments; (4) release the catalyst/gaseous phase rapidly through one of the ports with mass flow meter to mimic explosion technologies along with measuring mass loss from the system; (5) perform a controlled spray of a desired liquid, such as water, into the reactor along with release of steam from the reactor, through a port with attached mass flow meter, to aid in rapid cooling; (6) use an external jacket to heat the biomass from the walls of the reactors; (7) efficiently mix biomass-catalyst mixtures with viscosities varying from 10⁴ to 10⁸ cP; (8) use a novel mixing system that will scrape insoluble biomass-catalyst mixtures from the walls to avoid charring, which can lead to both insoluble reactant and heat transfer loss; and, (9) performed in a reactor, which is corrosion resistant and can be operated at high temperature and pressures.

Hastelloy alloys have the following properties: (1) Resistance to moderately to severely corrosive chemicals, (2) Resistant to high temperature, and (3) Resistant to high stress environments. Hastelloy C-22 and C-276 are highly temperature and corrosion resistant and have high nickel, molybdenum and chromium contents.

Flow patterns in the reactors are expected to alter continuously due changes in material behavior during conversion of insoluble reactants to soluble products. One of the possible effects of high solids loading is that stringy lignocellulosic materials, such as corn stover and switchgrass, are difficult to mix than corn cob and avicels. Stringy materials have a tendency to wrap around agitators and its blades.

In some embodiments, for high solids loading the impeller is modified from a magnetic drive to a self-sealing gland drive (i.e., high torque impeller for handling high solid loading). Although the extra heavy duty magnetic drive can handle up to torque 120 in-lb and gear box can handle up to torque 270 in-lb, the anchor impeller meets strong resistance due to the viscoelastic nature of the materials especially with high solid loading. Replaced it with self-sealing packing gland drive that can handle 270 lb-in torque that can be driven by 7.5 hp direct drive motor.

In some embodiments, for high solids loading, the reactants are added slowly,

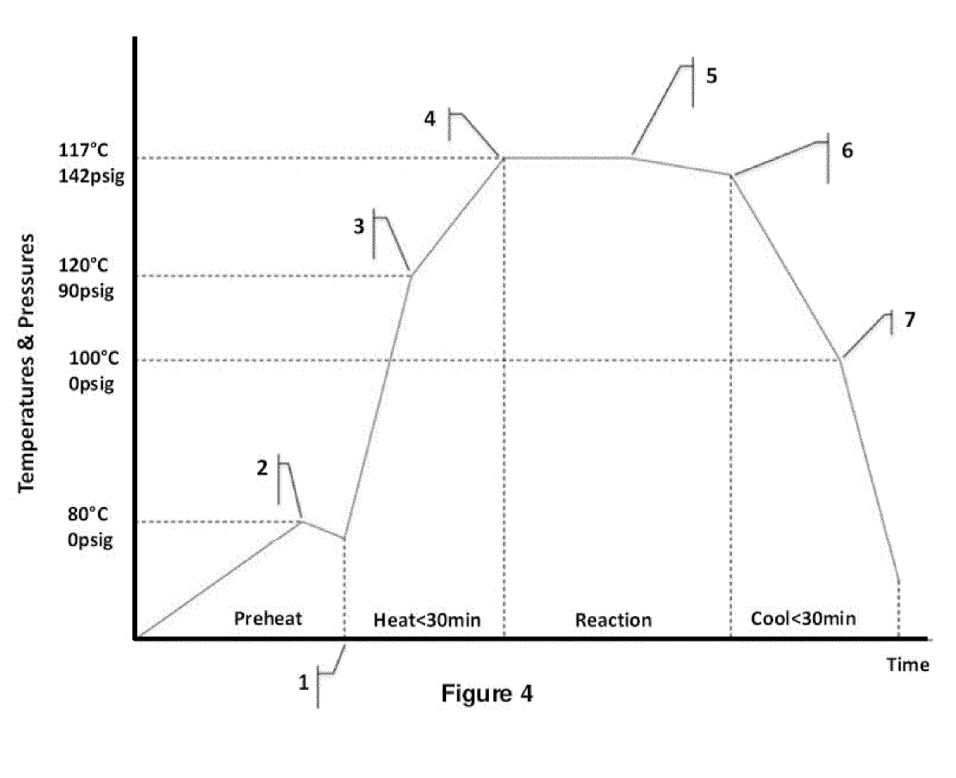

FIG. 4 shows the heating and cooling rates and set points which served as the basis for the process design. TCU heating to 80° C. (176° F. or 0 psig), steam injection heating to 130° C. (266° F. or 25.3 psig), and TCU heating to 180° C. (356° F. or 130 psig).

Xxx

The Andritz Helical Dryer can meet some of the required expectations of the “A Batch High-Pressure/Temperature Reactor to perform Homogenous and Heterogeneous Catalysis of Soluble or Insoluble Reactants in an Aqueous Phase” However, several updates including catalyst injection and release, water spray, vacuum line, and improved mixing with a system which continuously scrapes the wall will have to be incorporated into the system.

We used several off the shelf reactors that are proprietary materials for other companies. However, these reactors were not built or sold by the companies (Parr Instrument Company and Andritz Group) for thermochemical (homogenous and heterogenous) and enzymatic treatments of insoluble reactants, such as lignocellulosic biomass.

Xxx

Sustainable sources for generation of renewable liquid fuels are lignocellulosic and algal biomasses. Conversion of biomass to liquid fuels is challenging because it may have several polymers including complicated ones such as, lignin and glucan, which are insoluble in water and several other types of solvents, especially at room temperature. Catalysis of insoluble biomass is necessary to separate and depolymerize the polymers.

In some embodiments, the reactor vessel has one or more of the following advantages: Through this reactor, researchers can evaluate scalability issues associated with novel and/or established small scale thermochemical and/or enzymatic treatments. Through this reactor design, production scale facilities can be flexible in accepting and processing several feedstock types by performing multiple established thermochemical and enzymatic treatments. Faster temperature and pressure ramp rates using multiple chemical treatments will improve yields (e.g. sugar from lignocellulosic biomass) and reduce inhibitor (e.g. furfural and HMF from lignocellulosic biomass) yields. Also, for the case of lignocellulosic biomass, studies have indicated that lignin can also act as an inhibitor during fermentation. Performing hydrolysis of feedstocks to simpler sugars and separating lignin from biomass prior to fermentation will improve overall process yields and provide a possibility to vend lignin as a co-product.

In some embodiments, the reactor vessel has one or more of the following advantages: The present invention is highly valuable to biorefineries to evaluate several thermochemical and enzymatic treatments of multiple feedstocks at high solid loadings in a single reactor to produce and separate liquor with high concentration of product(s) and low concentration of inhibitory compounds. Facilitated by the improved mass and heat transfer in this design, the kinetics, concentrations, and yields of feedstock conversion will be more easily controlled and optimized at a higher level.

In some embodiments, the reactor vessel is capable of one or more of the following:
(1) continuously regulating nitrogen pressure in the impeller head at 5 psi above the reactor pressure to avoid misplacement of the agitator due to backpressure.
(2) inject catalyst (e.g. steam, ammonia, liquid catalysts, and/or solvents) into the pressure reactor through separate ports with mass flow meters while mixing the biomass in the reactor to establish efficient addition of liquid and gaseous catalysts.
(3) allow injection of preheated catalyst through a port with mass flow meter, for direct and rapid heat up.
(4) create a vacuum in the reactor to remove non condensables prior to heating or heat under vacuum to avoid high temperatures and degradation of products that can occur thereafter.
(5) ability to maintain a nitrogen overlay to avoid contact of flammable solvents with oxygen.
(6) automatic shutoff of the steam valve when the reactor reaches a pressure of 5 psi lower than the steam line itself.
(7) automatic opening of the steam valve once reactor pressure reaches 20 psi lower than steam line due to condensation of steam on contents in the reactor.
(8) release insoluble reactants rapidly through a discharge valve that can open under pressure to achieve explosion level treatments.
(9) release the gaseous phase rapidly through one of the ports with mass flow meter to mimic explosion technologies along with measuring mass loss from the system.
(10) perform a controlled spray of a desired liquid, such as water, into the reactor along with release of steam from the reactor, through a port with attached mass flow meter, to aid in rapid cooling.
(11) use an external jacket to heat the biomass from the walls of the reactors.
(12) efficiently mix biomass-catalyst mixtures with viscosities varying from 10⁴ to 10⁸ cP.
(13) corrosion resistance to acid and chlorides at high temperature and pressures.
(14) continuous addition of acids or other catalysts/solutes via injection ports.
(15) continuous data acquisition via LabVIEW VI suitable to obtain rapid heating and cooling profiles.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Design and Operation of “Tea-Cup” (“T-CuP”) Reactors for Heterogeneous Catalysis of Insoluble Solids

Pressure reactors are used extensively in petroleum, polymer, biofuels, pharmaceutical, and chemical processing industries. But very little information on reactor design and specifications has been published. In this study, we describe 10 and 210 L batch reactors designed to scale-up heterogeneous catalysis of insoluble solids in an aqueous phase. These reactors were designed, developed, and tested at Advanced Biofuels Process Demonstration Unit (ABPDU), Lawrence Berkeley National Lab (LBNL) to rapidly reach high temperatures (10 to 350° C.) and high pressures (0 to 1800 psig) and efficiently handle high concentrations of insoluble solids (up to 10,000 cP) in the aqueous phase substrate. Reactor vessels were built with Hastelloy C-22 to provide resistance to acidic and other corrosive chemical catalysts. To ensure improved mass transfer, the insoluble and solid catalysts should remain suspended in the aqueous phase throughout the reaction. Also, flow patterns in the reactors were expected to alter continuously due changes in material behavior during conversion of insoluble reactants to soluble products. Three different types of impellers: turbine, anchor, and helical impellers were selected to provide effective mixing and suspension of the solids in high to low viscosity aqueous phase substrates. The scale-up reactors were custom fitted with both direct (steam or solvent) and indirect (oil in an external dimpled jacket) heating. Direct heating is particularly useful for the scale-up of reaction chemistries that require rapid heating of reaction contents to target temperature. Other features of 210 L reactor include a knife gate valve that opens under pressure to simulate explosion-like studies, head space for solvent expansion, mass flow meters to measure the loss of solvent during venting process, etc. We were successfully able to commission the reactors with a fast heating rate of 3.5° C./minute and achieved a 95% overall mass balance closure. In this presentation, we will discuss the learnings from the reactor design, development, and commissioning of the reactors and also share the results from scale-up of heterogeneous catalysis of lignocellulosic biomass to produce cellulosic sugars and levulinic acid. The findings from this study will benefit researchers in industry and academia that are intending to scale-up reaction chemistries that require special considerations for improved mass and heat transfer and also intend to achieve 95+% mass balance closure at a scale-up level operation.

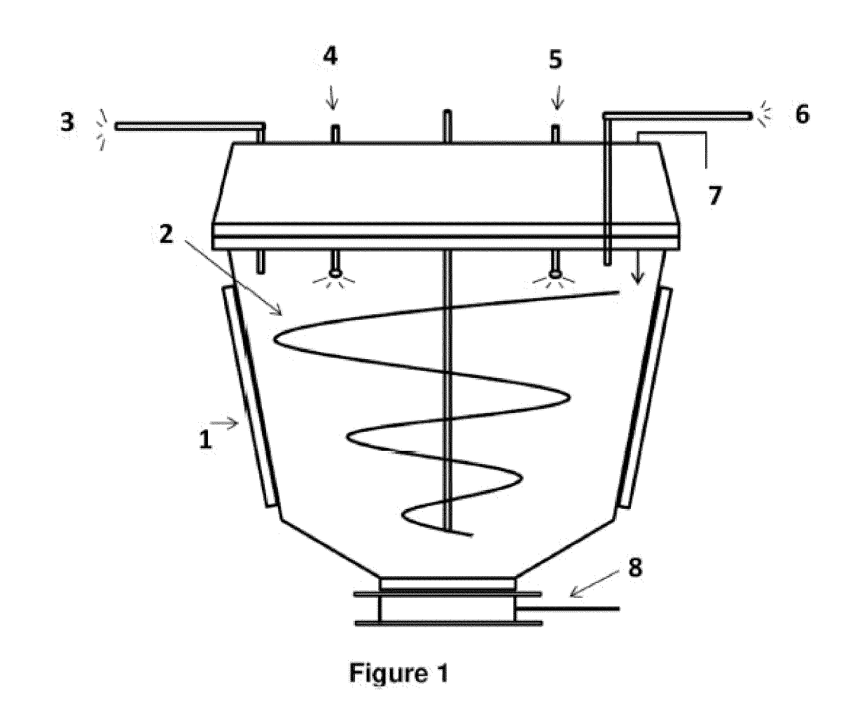

The design and results are shown in FIGS. 1-4.

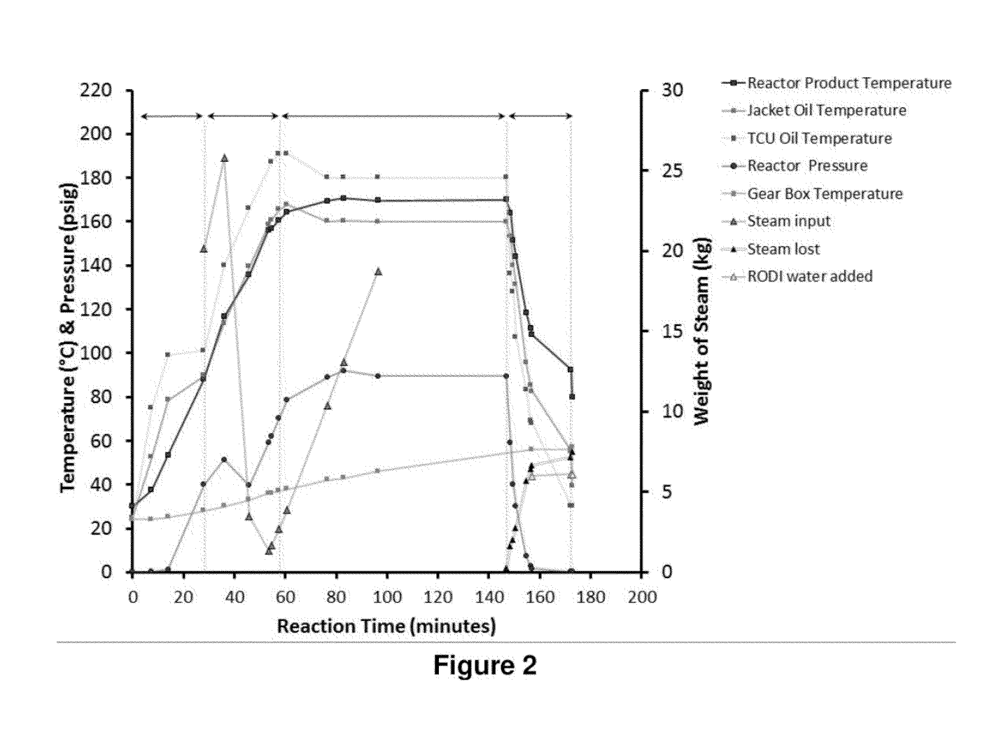

For FIG. 2, there is a fast heating rate of 3.50 C/minute. Process Test: temperature trends for several process points as well as reactor pressure. Through the use of steam injection and oil circulation, the reactor heating and cooling profiles performed comparably, relative to the design targets. The system is able to raise the temperature to the target value within 33 minutes, hold the temperature, and lower the process temperature in under 30 minutes.

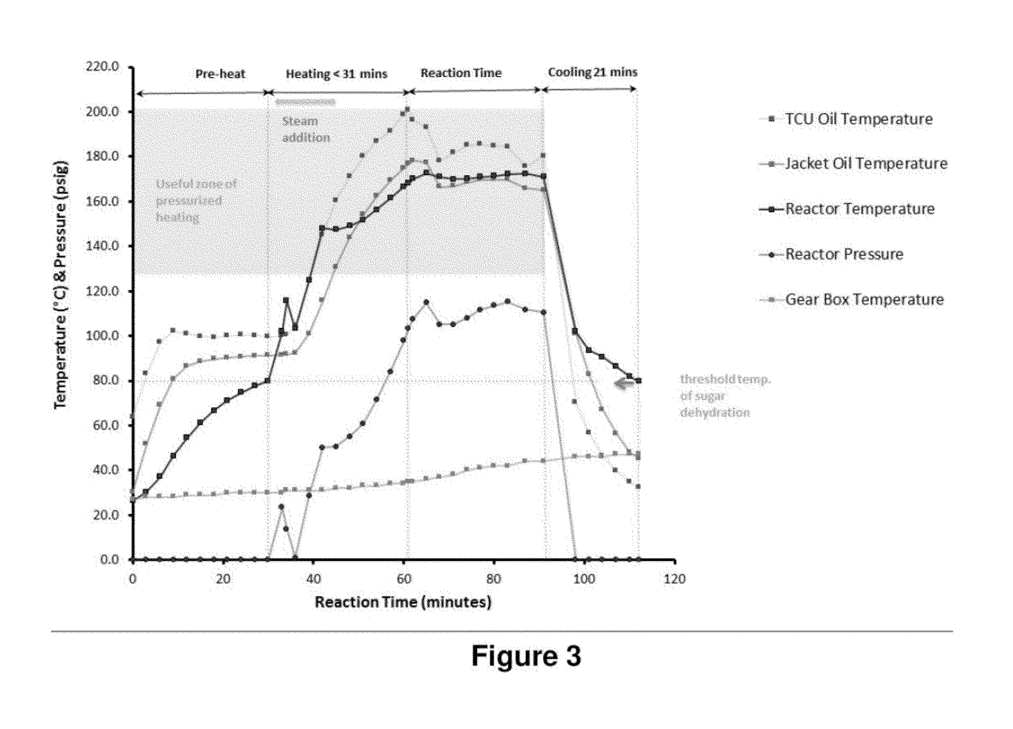

For FIG. 3, the reaction conditions are: 30 wt % corn stover loading, 1% w/w sulfuric acid (based on the dry biomass loading), temperature 170° C., time 30 mins, direct heating to TCU, and indirect heating to Steam. It is important to reach 80-170° C. at a faster rate to prevent the formation of furfural and HMF. Xylose degrades to HMF.

The reactors have the following advantages: Broad process compatibility, Well controlled, Corrosion resistant, High solids mixing, Meaningful heating rates and ranges, Pressure rated, Can open under pressure (Only for Andritz Pressure vessel), and Pumping of high solids & high viscosity (Only for Andritz Pressure Vessel).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A reactor vessel comprising a reaction chamber comprising an inner surface configured for performing multiple pretreatments, wherein the reactor vessel is configured for integration of multiple catalytic technologies in a single vessel for an insoluble feedstock at high solids loadings.

2. The reactor vessel of claim 1, wherein the reactor vessel is a single-stirred pressure reactor.

3. The reactor vessel of claim 1, wherein the reactor vessel comprises a working volume of at least 100 L.
